(19) United States
(12) Patent Application Publication
(10) Pub. No.: US 2018/0116875 A1
(43) Pub. Date: May 3, 2018

(54) Ski Goggle Lens Protector

(71) Applicant: Mary Kehoe, Boxford, MA (US)

(72) Inventor: Mary Kehoe, Boxford, MA (US)

(21) Appl. No.: 15/341,430

(22) Filed: Nov. 2, 2016

(51) Int. Cl. A61F 9/02

(52) U.S. Cl. CPC A61F 9/029

(57) ABSTRACT

The ski goggle lens protector is a ski goggle cover comprising of a single piece of oval shaped fabric, approximately 10½ inch by 12½ inch in size. It is then hemmed on the total perimeter with a braided elastic band inserted and drawn together at all edges to facilitate and secure a tight fit around the goggle to protect the goggle lens from scratches during storage.

This product generally relates to ski and snowboarding accessories. Specifically, the goggle lens protector is constructed to allow the user to be able to protect the goggle lens from normal scratches that might occur while being stored while attached to a typical ski helmet. It enables the user to conveniently leave the goggles on the helmet while being stored so the user can gear up without having to fumble with reattaching and readjusting the goggles prior to skiing or snowboarding.

At the end of a full day of skiing the goggle lens protector is a fast and convenient way to put goggles away safely without having to remove them from the ski helmet. When the ski day is done, rather than having to unhook the goggle straps that connect into the helmet slot of a typical ski helmet and storing the goggles in a separate bag, the goggle lens protector allows the user to keep the goggles attached to the helmet when stored away. When the next ski day comes, all the user has to do is slip the goggle lens protector off the attached goggles, slip the helmet on and hit the slopes. No need to reattach and readjust the goggles every time you start your skiing or boarding day. The goggle lens protector allows the user to simply slip the goggle lens protector on or off and go.

At the end of the ski day, remove the helmet, keeping the goggles attached, and slip the goggle lens protector over and around your goggles. The user is now ready to safely store both the helmet and the goggles together in the user's ski/boot bag or other.

### BRIEF DESCRIPTION OF THE VIEWS OF THE DRAWINGS, FIG. 1-4

FIG. 1 Drawing of a Goggle Lens Protector comprising of a single piece of oval shaped fabric approximately 10½ inches by 12½ inches in size. The outer edges are turned in and hemmed on the total perimeter and then a braided elastic band is inserted and drawn together at all edges.

FIG. 2 Drawing shows the goggle lens protector after the hem of the perimeter is stitched and the braided elastic band inserted and drawn together at all edges. This image shows the goggle lens protector ready for goggle coverage.

FIG. 3 Drawing is a side view of a goggle lens protector attached to a typical ski or snowboard helmet.

FIG. 4 Drawing is a top view of the goggle lens protector attached to a typical ski or snowboard helmet.

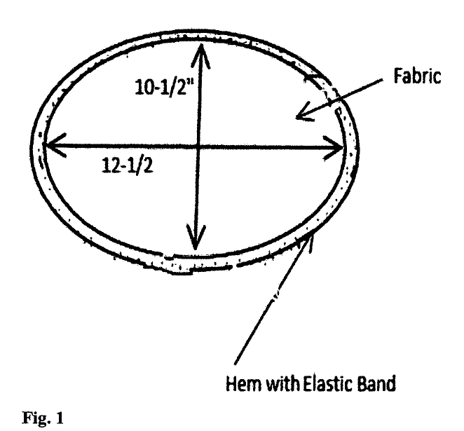

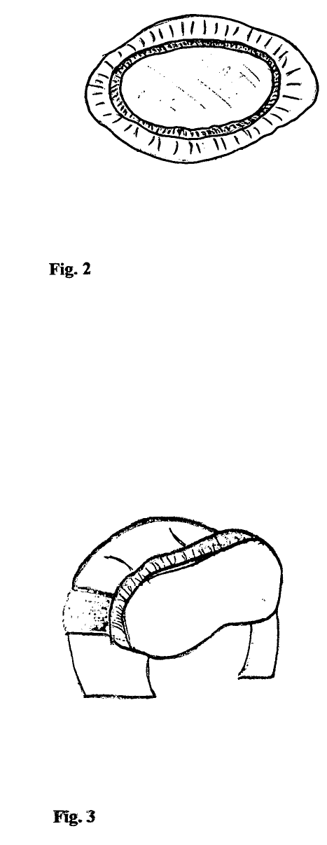

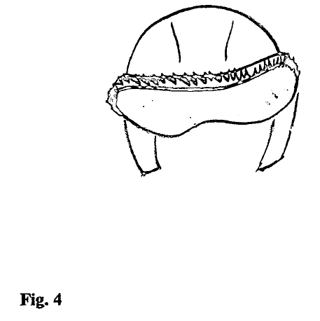

1. The goggle lens protector protects the goggle lens from normal scratches and dings in storage while attached to the user's helmet.

2. The goggle lens protector allows the user to quickly and conveniently store both the helmet and the goggles together without removing the goggle from a typical ski or snowboard helmet by using nonabrasive fabric used in the manufacturing of the goggle lens protector and slipping it on, around and over the goggles prior to storage.

3. The goggle lens protector saves time when gearing up for skiing and packing up after a day on the slopes. When gearing up for the day, the goggle lens protector enables the user to slip the goggle lens protector off the goggles while still attached to the helmet thus eliminating the steps of reattaching and readjusting the goggles every time the user goes out for the day. After skiing or snowboarding, it eliminated the steps of taking the goggles off the helmet and storing them in a separate goggle bag or other for storage making for a faster pack-up.
